(19) United States
(12) Patent Application Publication
(10) Pub. No.: US 2025/0114106 A1
(43) Pub. Date: Apr. 10, 2025

(54) Targeting Device

(71) Applicant: Createc GmbH & Co. KG, Friedrichshafen (DE)

(72) Inventors: Dominik MATHYS, Stettlen (CH); Berthold VOGEL, Schwieberdingen (DE); Andris KOPPLIN, Konstanz (DE)

(73) Assignee: Createc GmbH & Co. KG, Friedrichshafen (DE)

(21) Appl. No.: 18/729,866

(22) PCT Filed: Jan. 17, 2023
(86) PCT No.: PCT/EP2023/051024

§ 371 Date: Jul. 17, 2024

(30) Foreign Application Priority Data
Jan. 18, 2022 (EP) 22152076.0

(51) Int. Cl. A61B 17/17; A61B 17/00

(52) U.S. Cl. CPC A61B 17/1717; A61B 2017/00862

(57) ABSTRACT

The present application relates to a targeting device for developing a securement of a prosthesis with a bone securement element on a bone, comprising holding means for positioning the prosthesis in a first axis, a mounting-guide receptacle forming a guide opening which is oriented along a second axis and penetrates the targeting device and is developed to receive a protective sleeve, and arresting means which comprises the at least one elastically deflectable finger which can be axially and/or radially deflected with respect to the second axis, and wherein the first axis and the second axis intersect, wherein the at least one finger projects into the guide opening.

### CROSS-REFERENCE TO RELATED APPLICATION

The present application is a 371 National Phase Application of International Patent Application No. PCT/EP2023/051024, filed Jan. 17, 2023, which claims priority to European Patent Application No. 22152076.0, filed Jan. 18, 2022, the entirety of each of which is incorporated herein by reference.

### FIELD OF APPLICATION

The present application relates to a targeting device for developing a securement of a prosthesis, in particular of an intramedullary nail, on a bone with a bone securement element.

### BACKGROUND

Such targeting devices are known in prior art in diverse implementations and serve for the precise targeted positioning of a bone securement element in a corresponding recess of a prosthesis, for example of an intramedullary nail, in order to connect it with the bone and fix the prosthesis firmly with the bone and anchor it thereto. Such targeting devices are widespread to stabilize bones (for example femurs or tibias) after a fracture.

For this purpose the utilized prostheses comprise a recess through which the bone securement element can be introduced in order to anchor the prosthesis, respectively the intramedullary nail, to the bone. Corresponding to this recess, a recess can be introduced by means of a tool, wherein the corresponding recess is precisely introduced onto the recess in the prosthesis by means of the targeting device with a guide opening. To prevent damage of the surrounding tissue when employing the targeting device during the introduction of the corresponding recess, the tissue is held at a distance from the tool.

Apart from tissue spreaders, protective sleeves are also used which shield the tool during the introduction of the corresponding recess into the bone.

### SUMMARY

The present application addresses the problem of proposing an expediently improved targeting device. Damages to the surrounding tissue when introducing the corresponding recess into the bone through the tool are to be prevented by means of a protective sleeve, wherein manipulating the targeting device is to be simple and reliable.

These problems are resolved by the targeting device of the present application.

The targeting device for developing a prosthesis securement, in particular of an intramedullary nail, on a bone comprises holding means for positioning the prosthesis, in particular the intramedullary nail, in a first axis; at least one mounting-guide receptacle forming at least one guide opening, which is oriented along a second axis and drives through the targeting device and is developed to receive the at least one protective sleeve, and at least one arresting means which comprises at least one elastically deflectable finger which can be axially and/or radially deflected with respect to the second axis. The first axis and the second axis intersect wherein it is further provided that the at least one elastically deflectable finger projects into the guide opening. The at least one elastically deflectable finger is configured to fix the protective sleeve in position along the second axis.

The present application is based on the concept of fixing the protective sleeve within the guide opening by the at least one elastically deflectable finger. The protective sleeve therefore does not need to be held by medical personnel and there is furthermore no risk of the protective sleeve falling unintentionally out of the guide opening.

It should be noted here that the targeting device can comprise more than one mounting-guide receptacle forming a guide opening. The particular guide openings can each be oriented along a second axis, wherein the second axes are spaced apart from one another. The second axes can be oriented parallel with respect to one another or they can intersect in a common intersection point. The included angle formed by the second axes is between 30° and 60°.

The at least one elastically deflectable finger can come into effective contact with the protective sleeve set into the guide opening and effect a frictional connection with the protective sleeve. When inserting the protective sleeve into the guide opening the at least one elastic finger is axially and/or radially deflected and exerts a fixing holding force onto the protective sleeve.

Through the appropriate layout of the rigidity of the at least one elastic finger the holding force can be dimensioned, and in this way the holding force can be adapted to the particular application or to preferences of the user.

The guide opening and/or the protective sleeve are preferably cylindrical and the guide opening drives completely through the targeting device. The guide opening is preferably developed such that the protective sleeve can be guided through smoothly, for example by means of a clearance fit.

In another embodiment, the arresting means comprises a connector. The connector can be a hollow cylinder with a through-hole, wherein the through-hole is preferably adapted in form and/or dimension to the guide opening or is equal to it, and moreover can form a section of the guide opening.

The through-hole can have a polygonal, circular or octagonal cross section, wherein the through-hole is developed such that at least one guide protective sleeve can be guided therethrough. The octagonal cross section can make it feasible for the at least one guide protective sleeve to be guided through in at least two positionings parallel and spaced apart from the second axis.

The at least one elastic finger projects away from the arresting means or the connector radially inwardly or radially inwardly and axially. The connector can be fastened in any desired way on the targeting device, wherein the through-hole and the guide opening are disposed coaxially in the second axis.

In some embodiments, the arresting means or the connector can completely be disposed in the mounting-guide receptacle of the targeting device. The connector may be disposed in the mounting-guide receptacle such that it cannot be lost. The mounting-guide receptacle preferably comprises a widening in the manner of a cylindrical (fastener) countersink into which the connector can be set. The connector is held in the mounting-guide receptacle oriented coaxially to the guide opening.

It has, moreover, been found to be advantageous if an annular space is formed in the mounting-guide receptacle, and that the at least one finger can project freely movably into the annular space. The annular space may be developed frontally with respect to the arresting means or the connector, respectively, and may be encompassed in the form of a U on the one side by the mounting-guide receptacle and, on the other side, by the connector.

In some embodiments, a flushing channel terminates into the annular space. Through the flushing bore a cleansing agent can be introduced into the annular space in order to cleanse the targeting device before or after use.

In some embodiments, the flushing channel terminates into the annular space in a direction oriented tangentially to the second axis whereby in the cleansing process the annular space can be effectively flushed throughout with a cleansing agent.

In some embodiments, the connector has a U-shaped cross section. The connector comprises an inner flank, an outer flank and a middle flank, wherein the middle flank connects the inner flank and the outer flank. Due to the U-shaped physical form of the connector, the annular space can be developed by the flanks of the U-shaped connector.

A connection means can be provided with which the arresting means can be secured in the mounting-guide receptacle under force closure, form closure and/or material closure. In some embodiments, the inner flank encompasses the at least one finger.

In some embodiments, at least two, three, or more, separate fingers are provided. The separate elastically deflectable fingers can be disposed so as to be distributed over the periphery about the second axis and spaced apart from one another and project in the form of spokes or combs into the guide opening. Furthermore, along the second axis at least two elastic fingers can be disposed. Through the number of elastic fingers, furthermore, the holding force for the protective sleeve can be laid out.

In some embodiments, the separate and elastic fingers are disposed circumferentially symmetric with respect to the second axis. The circumferentially symmetric disposition of the separate fingers enables centering the protective sleeve in the guide opening.

In some embodiments, at least one elastically deflectable finger comprises a contact region at a free end. The contact region can be configured to generate the highest feasible holding force and the highest feasible static friction. The contact region can be developed of an appropriate material and/or can comprise an appropriately equipped surface which has the highest feasible friction coefficient.

In some embodiments, the contact region can form an angle with respect to an orientation of the at least one finger, wherein the contact region is configured to rest in surface contact on the protective sleeve.

In the following an embodiment example of the targeting device as well as further developments of the targeting device are described in detail with reference to the accompanying drawings. Therein depict:

### BRIEF DESCRIPTION OF THE DRAWINGS

FIGS. 1a-1c a perspective representation of a targeting device holding means which position a prosthesis in a first axis and with a protective sleeve which is secured in a second axis by arresting means,

FIGS. 2a-2c various views of the arresting means according to FIG. 1,

FIGS. 3a-3c various views of a second embodiment of the arresting means according to FIG. 1,

FIGS. 4a-4c various views of a third embodiment of the arresting means according to FIG. 1,

FIGS. 5a-5c various views of a fourth embodiment of the arresting means according to FIG. 1,

FIGS. 6a-6d various views of a fifth embodiment of the arresting means according to FIG. 1, and

FIG. 7 a sixth embodiment of the arresting means according to FIG. 1.

In the following identical or functionally identical structures in an embodiment example are identified with identical reference symbols. For the sake of greater clarity not all identical or functionally identical structures are provided with reference numbers in the individual Figures.

### DETAILED DESCRIPTION

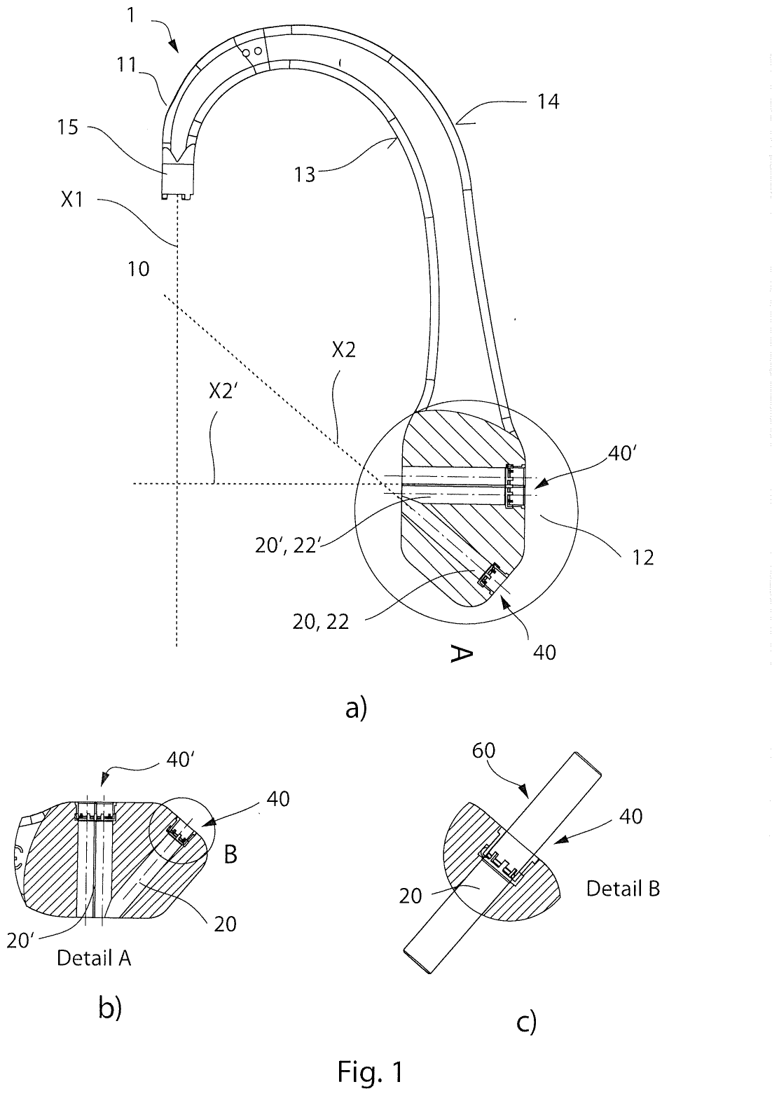

FIG. 1a shows a depiction of a targeting device 1 according to an embodiment.

The targeting device 1 is developed in the shape of a bow and comprises a first end region 11 and a second end region 12 and can be comprised of one part or several parts denoted overall as main body 10. The targeting device 1 comprises further a proximal side 13 and a distal side 14, wherein the proximal side 13 rests on a concave region of the bow-shaped targeting device 1 and the distal side 14 rests on a convex region opposite the concave region.

In the first end region 11 holding means 15 for positioning a prosthesis 8, in particular an intramedullary nail, not shown, are provided, wherein the prosthesis 8 can be held in an orientation along a first axis X1. The holding means 15 can establish a detachable connection with the prosthesis 8.

In the second end region 12 is developed at least one mounting-guide receptacle 20 forming at least one guide opening 22, wherein the mounting-guide receptacle 20 completely penetrates the targeting device 1 or the main body 10, respectively, and the mounting-guide receptacle 20 connects the proximal side 13 with the distal side 14. The mounting-guide receptacle 20 is oriented in a second axis X2, wherein, as depicted in the embodiment example according to FIG. 1, several mounting-guide receptacles 20 are provided. For the sake of clarity, in the accompanying Figures the further mounting-guide receptacle 20, the further guide opening 22 and the further second axis X2 are marked by “′”

The first axis X1 and the second axis X2 possess a common intersection, wherein the angle of intersection on the side facing the targeting device 1 should be approximately between 45° and 135°.

Into the mounting-guide receptacle 20 or into the guide opening 22, respectively, can, as shown inter alia in FIGS. 1c, 2a, 3a, 4a, 5a, a protective sleeve 60 be set in or be guided through, respectively, wherein the protective sleeve 60 can be set into the mounting-guide receptacle 20 and the guide opening 22 along the second axis X2 such that it runs smoothly. For this purpose, the shapes of the protective sleeve 60 and the guide opening 22 correspond to one another.

In a manner not shown, into the mounting-guide receptacle 20′ or into the guide opening 22′, respectively, can also analogously be inserted or guided through two protective sleeves 60 or one protective sleeve 60 in different positionings.

The mounting-guide receptacle 20 and/or the guide opening 22 can have a circular or octagonal cross section, wherein further the guide opening 22 determines the alignment of the protective sleeve 60 in the second axis X2. The guide opening 22 has a diameter D22 and the protective sleeve 60 has a diameter D60, wherein, for forming a clearance fit, the diameter D60 is minimally smaller than the diameter D22.

The targeting device 1 further comprises arresting means 40 which is configured to fix the protective sleeve 60 in position in the mounting-guide receptacle 20 to avoid it inadvertently or unintentionally falling out.

The arresting means 40 according to FIGS. 1 to 7 comprises at least one elastic finger 50 which, with respect to the second axis X2, can be axially and/or radially deflected and can come into effective contact with the at least one protective sleeve 60. The at least one elastically deflectable finger 50 projects freely movably into the guide opening 22 and during the insertion of the protective sleeve 60 into the guide opening 22 can carry out an evasive elastic motion and subsequently press with a holding force against the protective sleeve 60 in order to fix it in a desired position in the guide opening 22.

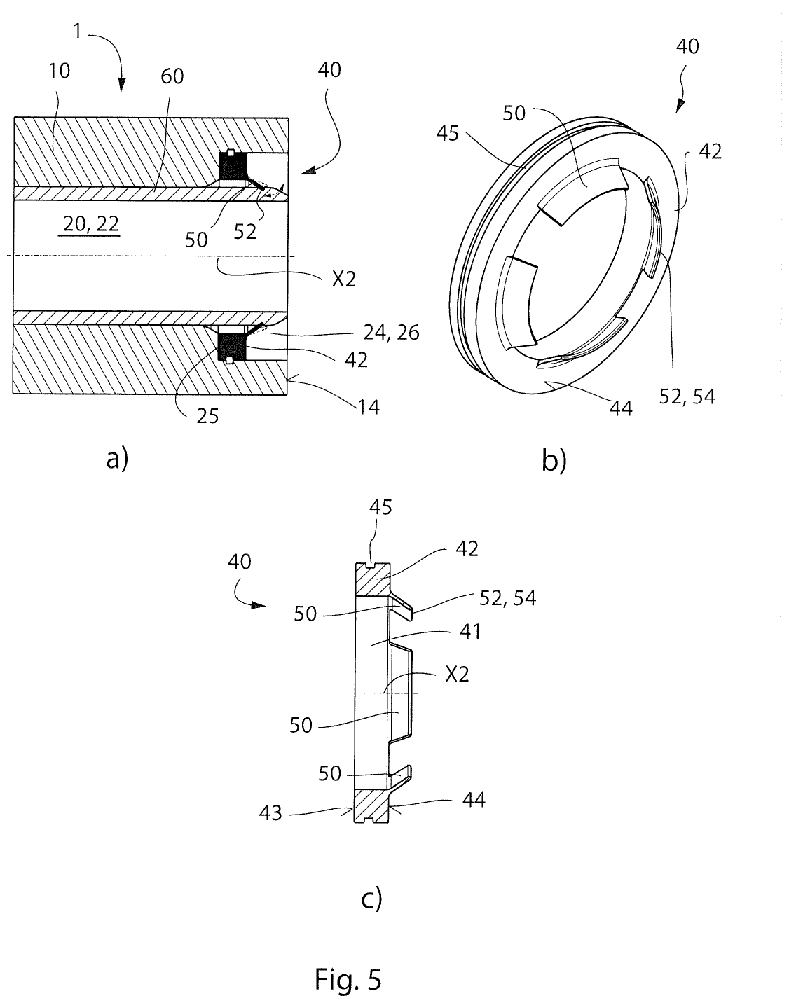

With respect to the second axis X2, the at least one elastic finger 50 projects radially or radially and axially into the guide opening 22. In the non-deflected state of the finger 50 a free end 52 of the particular finger 50 is located within the guide opening 22. It should be noted here that in the accompanying Figures the fingers 50 are depicted in the non-deflected state for reasons of simplified depiction. Only in FIG. 5a a deflection motion is indicated by means of dashed lines as well as in conjunction with a double arrow.

The free end 52 can comprise a contact region 54 which has increased friction coefficient properties.

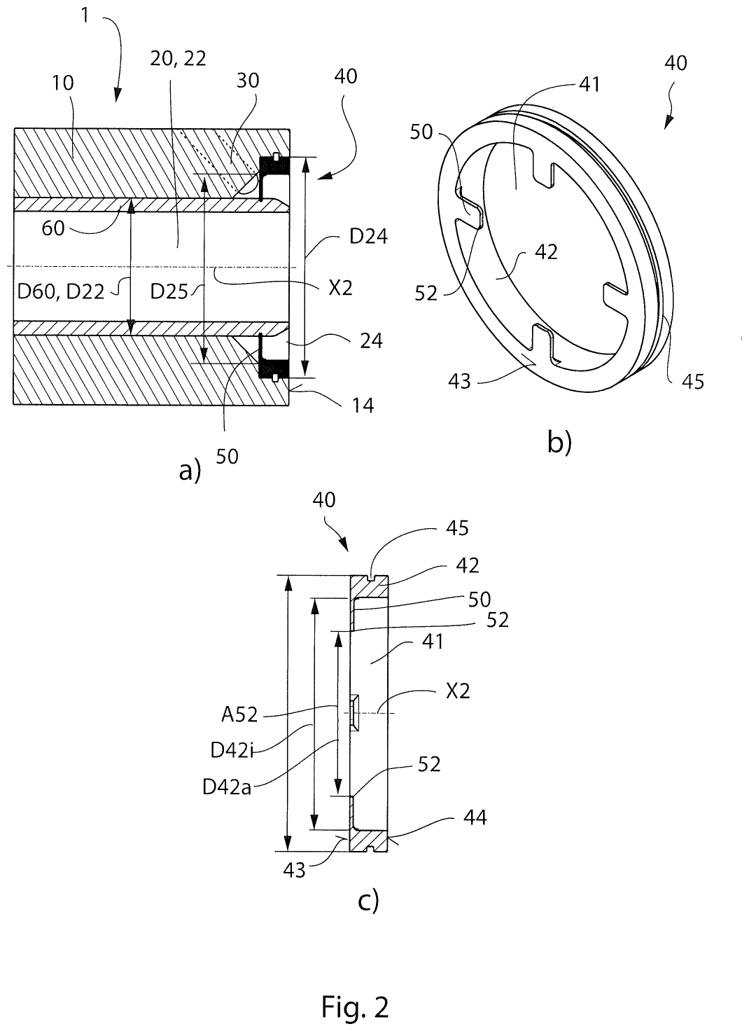

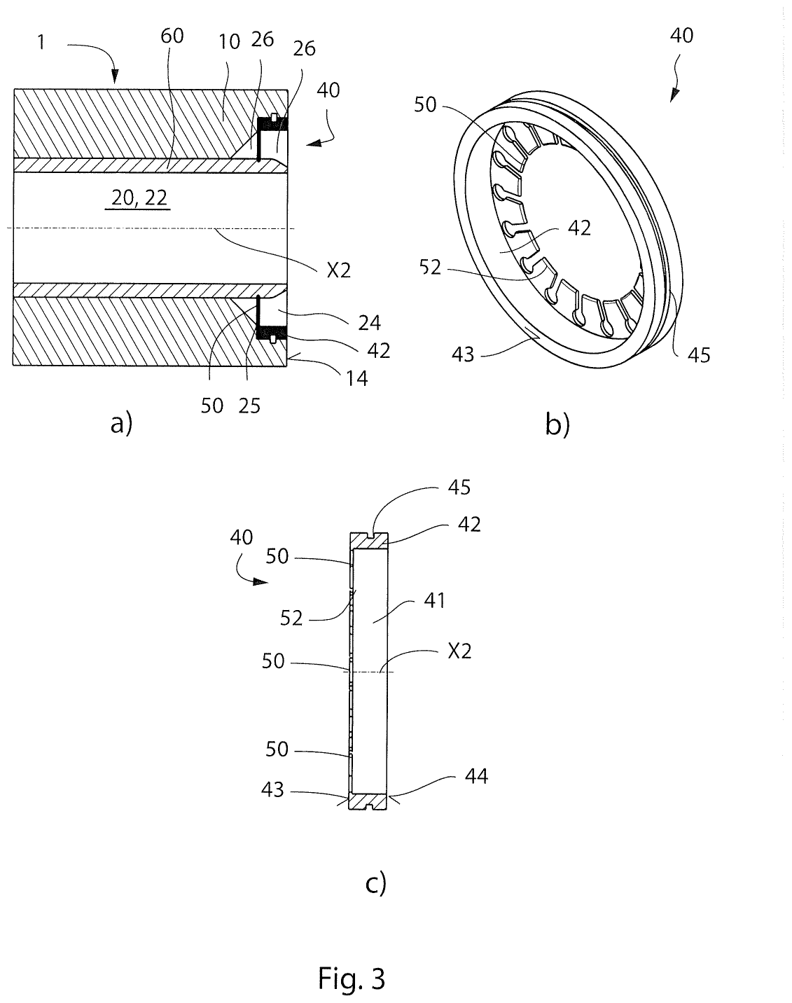

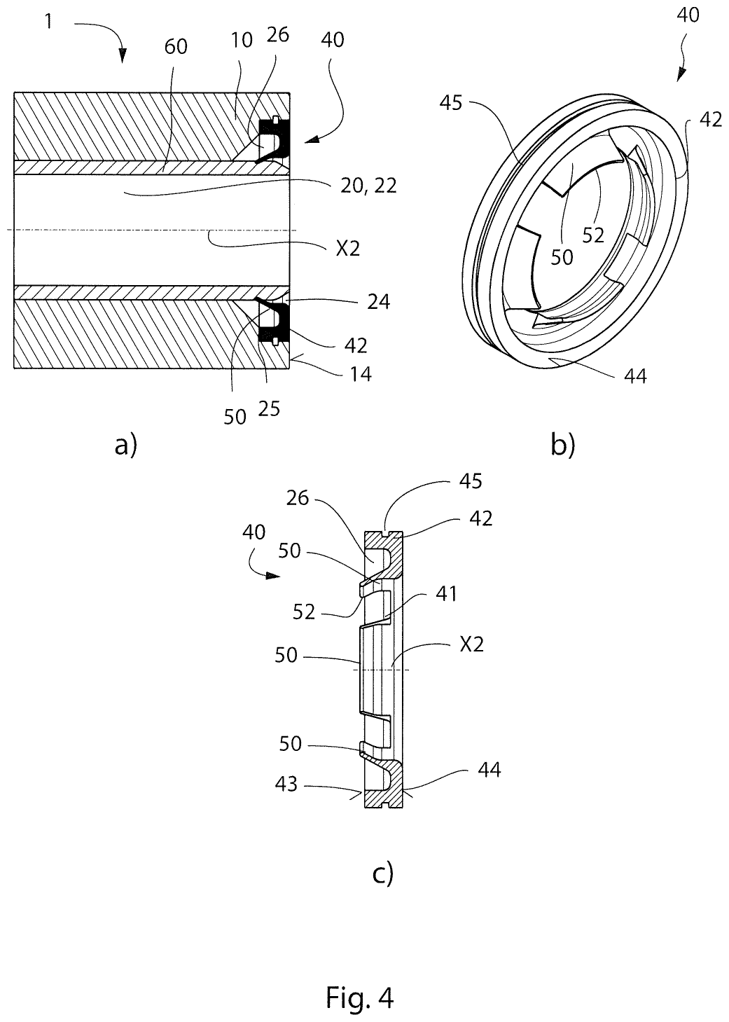

According to the embodiment examples according to FIGS. 2 and 3 the elastically deflectable fingers 50 project, for example, radially into the guide opening 22 while the elastic fingers 50 according to the embodiment examples after FIGS. 4 and 5 are oriented at an inclination angle of approximately 45° toward the second axis X2 and accordingly project radially and axially into the guide opening 22.

As shown in the depicted embodiment examples, the arresting means 40 can comprise a connector 42 which can be set into the mounting-guide receptacle 20. From the connector 42 project the elastic fingers 50 free-standingly with respect to the second axis X2 in the radial direction or in the radial and axial direction in the direction of the second axis X2.

The mounting-guide receptacle 20 can comprise on the proximal side 13 and/or on the distal side 14 a widening 24 which is configured to receive the connector 42. The widening 24 has a diameter D24, wherein the diameter D24 is greater than the diameter D22.

The connector 42 has an outer diameter D42a and the through-hole 41 of the connector 42 has an inner diameter D42i. The outer diameter D42a corresponds to the diameter D24 of the widening 24 and the inner diameter D42i is greater than the diameter D60 of the protective sleeve 60.

The connector 42 can be held in the mounting-guide receptacle 20 so as to be secured by connection means under force closure, form closure and/or material closure. For example, the disposition of the connector 42 in the mounting-guide receptacle 20 under material closure can be advantageous in order to prevent impurities from penetrating into a cut-in site between the connector 42 and the main body 10 of the targeting device 1.

For this purpose the connector 42 can comprise a cut-in groove 45 into which sealing means and/or an adhesive agent can be introduced.

The widening 24 can further comprise an abutment 25 developed as a ledge by which the position of the connector 42 in the mounting-guide receptacle 20 can be determined. One of the front faces 43, 44 of the connector 42 can come to rest in contact with the abutment 25.

With reference to FIG. 2a, it is further evident that in the mounting-guide receptacle 20 an annular space 26 is developed adjacent to the widening 24, into which the at least one elastic finger 50 can immerse.

The annular space 26 can be formed by a conical section which connects the widening 24 or the abutment 25, respectively, with the guide opening 22. The greatest diameter D25 of the conical annular space 26 or of a cylindrical annular space 26 should be greater than the inner diameter D42i in order to enable the unhindered deflection motion of the elastic finger 50.

In FIGS. 2 to 7 various embodiments of the previously generally described arresting means 40 are depicted, wherein the arresting means 40, described in the following in detail, have the above described properties in common.

The arresting means 40 according to FIG. 2 comprises several elastic fingers 50, disposed circumferentially symmetric about the second X2, which project radially in the direction of the second axis X2 into a through-hole 41 of connector 42 or into the guide opening 22, respectively. The distance A52 between two diametrically disposed free ends 52 is less than the diameter D60.

In FIG. 2 is further indicated that a flushing channel 30 can be provided which terminates into the annular space 26. The flushing channel 30 is configured to introduce a cleansing agent into the annular space 26 in order, for example, to cleanse or sterilize the targeting device 1 after it has been used. The flushing channel 30 can terminate into the annular space 26 in a direction oriented tangentially with respect to the second axis X2, whereby the annular space 26 during the cleansing can be effectively flushed throughout.

The arresting means 40 according to FIG. 3 is developed analogously to the arresting means 40 according to FIG. 2, wherein a multiplicity of elastic fingers 50 is provided in close-spacing. The spacing between the elastic fingers 50 in the circumferential direction is less than the width of the elastic fingers 50. A clearance between the elastic fingers 50 comprises a relief counterbore on the side facing away from the free end 52. The relief counterbore can reduce the notch effect of the notch-form clearance.

FIG. 4 shows an arresting means 40 with U-shaped cross section. The arresting means 40 is implemented comparably to the already introduced arresting means 40 according to FIGS. 2 and 3. However, here the connector 42 is developed of U-shape in cross section and comprises an inner flank, an outer flank and a middle flank. The middle flank connects the inner flank and the outer flank, wherein the flanks encompass, at least in sections, the annular space 26.

The inner flank forms the elastic finger 50 whereby an especially compact construction of the arresting means 40 can be accomplished.

A further embodiment of the arresting means 40 can be found in FIG. 5, wherein the elastic fingers 50 project from a front face 43, 44 of the annular connector 42.

The connector 42 forming the arresting means 40 can be inserted in any desired orientation into the mounting-guide receptacle 20, wherein the elastically deflectable fingers 50 can point in both directions with respect to the second axis X2.

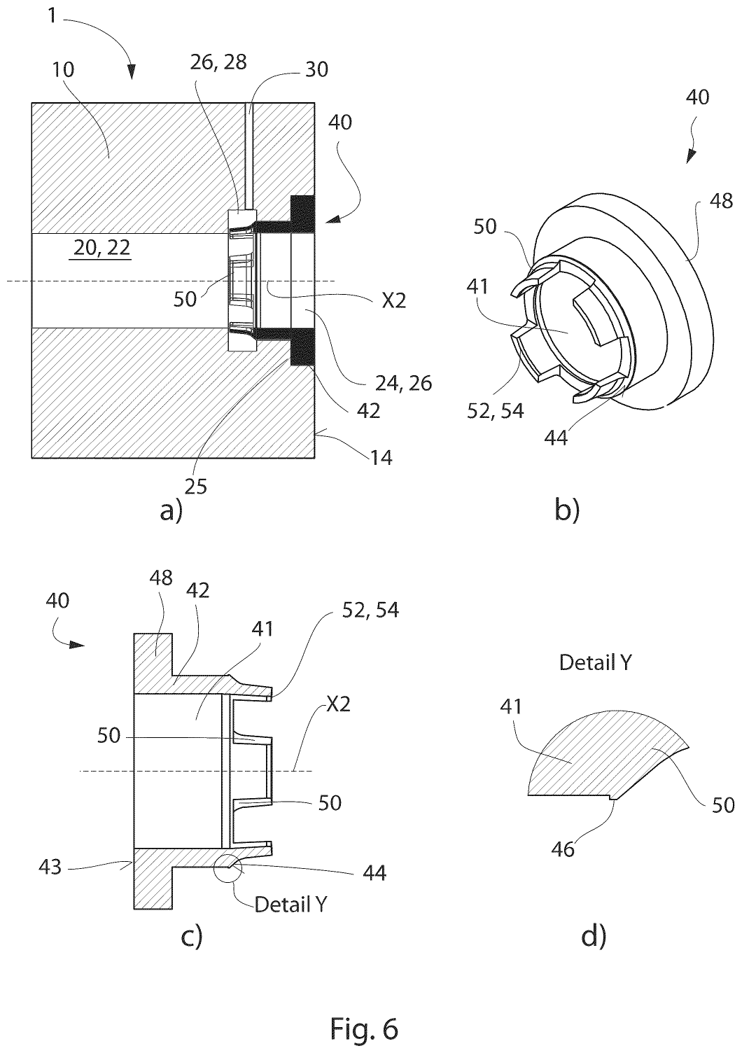

A fifth embodiment of the arresting means 40 and a further development of the main body 10 is shown in FIG. 6. The main body 10 comprises in the widening 24 an undercut 28 which is spaced apart from the distal 14 and/or proximal side 13. The undercut 28 can form the annular space 26. The deflectable fingers 50 project freely movably into the annular space 26 and in this way can execute unhindered deflection motions.

As is discernible in FIG. 6a in a manner comparable to that already described, a flushing channel 30 can be provided which terminates into the annular space 26.

The arresting means 40 according to FIGS. 6a to 6d is implemented comparably to the already introduced arresting means 40 according to FIGS. 4 and 5. However, here the connector 42 is developed to be more elongated in the second axis X2 and the elastic finger 50 projects from a front face 43, 44 of the connector 42. The opposing front face 43 or 44 comprises a flange section 48 which can cooperate with the abutment 25 in order to fix the position of the arresting means 40 in position in the second axis X2 in the mounting-guide receptacle 20.

In the detail depiction according to FIG. 6d can be discerned that the connection means of the arresting means 40 can comprise at least one projection 46 to hold the arresting means 40 under force closure in the mounting-guide receptacle 20. The projection 46 can be developed as a circumferential flange and is configured to secure the arresting means 40 clamped through an interference fit in the mounting-guide receptacle 20. It is also feasible to provide several tooth-form projections 46 distributed over the periphery.

The projection 46 is disposed adjacent to the deflectable fingers 50. In the depicted embodiment example the projection 46 is developed along the second axis X2 in the region from which the finger 40 projects, or stated more precisely, adjacent to the front face 43, 44 from which the at least one finger 50 projects.

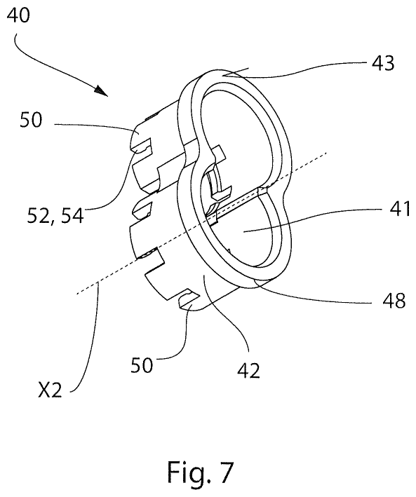

FIG. 7 shows a sixth exemplary embodiment of the arresting means 40 according to FIG. 1b which there [in this FIG. 1b] is denoted as reference number 40′. In contrast to the arresting means 40 depicted in FIG. 6, the arresting means 40 according to FIG. 7 comprises a through-hole 41 that is figure 8-shaped in cross section which is intended to enable for a (not shown) protective sleeve 60 to be inserted and secured in two positionings parallel and spaced apart from the second axis X2′. The through-hole 41, figure 8-shaped in cross section, can be developed such that the two circles framing the cross sectional-area intersect in one or two points of intersection. The framed circles can also be disposed spaced apart from one another. In such case the two protective sleeves 60 can be secured simultaneously through the arresting means 40.

### LIST OF REFERENCE SYMBOLS

1 Targeting device
8 Prosthesis
10 Main body
11 First end region
12 Second end region
13 Proximal side
14 Distal side
15 Holding means
20, 20 Mounting-guide receptacle
22, 22′ Guide opening
24 Widening
25 Abutment
26 Annular space
30 Flushing channel
40, 40′ Arresting means
41 Through-hole
42 Connector
43 First front face
44 Second front face
45 Cut-in groove
46 Projection
48 Flange
50 Finger
52 Free end of 50
54 Contact region
60 Protective sleeve
X1 First axis
X2 Second axis
A52 Distance
D22 Diameter of 22
D24 Diameter of 24
D25 Diameter of 25
D42a Outer diameter of 42
D42i Inner diameter of 42
D60 Diameter of 60

1. A targeting device for developing a securement of a prosthesis with a bone securement element on a bone, comprising:
a hold that positions the prosthesis in a first axis,
a mounting-guide receptacle forming a guide opening which is oriented along a second axis and penetrates the targeting device and receives a protective sleeve, and
a connector which comprises at least one elastically deflectable finger which can be axially and/or radially deflected with respect to the second axis,
wherein the first axis and the second axis intersect,
wherein the at least one finger projects into the guide opening.

2. The targeting device of claim 1, wherein the connector comprises a cylindrical connector.

3. The targeting device of claim 2, wherein the connector is disposable in the mounting-guide receptacle.

4. The targeting device of claim 1, wherein an annular space is developed in the mounting-guide receptacle and the at least one finger can freely movably project into the annular space.

5. The targeting device of claim 1, wherein a flushing channel terminates into the annular space.

6. The targeting device of claim 5, wherein the flushing channel terminates into the mounting-guide receptacle in a direction oriented tangentially to the second axis.

7. The targeting device of claim 1, wherein the connector is U-shaped in cross section.

8. The targeting device of claim 1, wherein the connector further comprises a projection via which the connector can be secured in the mounting-guide receptacle under force closure, form closure and/or material closure.

9. The targeting device of claim 1, wherein the connector is fabricated of a synthetic material.

10. The targeting device of claim 1, wherein several separate fingers are provided.

11. The targeting device of claim 10, wherein the separate fingers are disposed circumferentially symmetrical to the second axis.

12. The targeting device of claim 10, wherein at least one separate finger comprises a contact region at a free end.

13. The targeting device of claim 12, wherein the contact region of the at least one finger is angled such that it is in surface contact on the protective sleeve.
